(19) United States
(12) Patent Application Publication
(10) Pub. No.: US 2004/0024416 A1
(43) Pub. Date: Feb. 5, 2004

(54) Implantable braided stroke preventing device and method of manufacturing

(76) Inventors: Ofer Yodfat, Modi'in (IL); Ygael Grad, Tel Aviv (IL)

Correspondence Address: Anthony Castorina, G E Ehrlich, Suite 207, 2001 Jefferson Davis Highway, Arlington, VA, 22202, US

(21) Appl. No.: 10/311,876

(22) PCT Filed: Jul. 9, 2001
(86) PCT No.: PCT/IL01/00624

(30) Foreign Application Priority Data
Jul. 17, 2000 (IL) 137326

(51) Int. Cl. A61F 2/06

(52) U.S. Cl. 606/200; 623/1.15; 623/1.53

(57) ABSTRACT

An implantable deflecting device for positioning in the vicinity of an arterial bifurcation for causing embolic material flowing toward a first branch of the bifurcation to be deflected into the second branch of the same bifurcation. The device comprises a deflecting filtering element suitable to deflect the flow of embolic material flowing toward said second branch, while filtering the blood flowing toward said first branch. The device comprising a braided tubular body having a contracted state with a first diameter, and an expanded state having a second diameter greater than said first diameter.

### FIELD OF THE INVENTION

The present invention relates to implantable braided stroke preventing devices, and more specifically is concerned with a device for reducing the risk of embolic material entering into the internal carotid artery of an individual.

### BACKGROUND OF THE INVENTION

A major portion of blood supply to the brain hemispheres is by two arteries, referred to as common carotid arteries (CCA), each of which branches off, or bifurcates as the term is at times used, into a so called internal carotid artery (ICA) and an external carotid artery (ECA). Blood to the brain stem is supplied by two vertebral arteries.

Stroke is a leading cause of disability, death and health care expenditure. It is the second most common cause of death worldwide, exceeded only by heart disease, and the third most common cause in the US [Heart and Stroke Statistical Update. Dallas, Tex: American HeartAssociation; 2000].

Stroke is caused either due to ischemia-infarction or intracranial hemorrhage. Infarction constitutes 85 to 90 percent of the total group in western countries [Sacco RL, Toni D, Mohr JP. Classification of ischemic stroke. In: Barnett HJM, Mohr JP, Stein BM, Yatsu FM. and editors. Stroke: Pathophysiology, diagnosis and management. 3rd Ed. New York: Churchill Livingstone; 1998, PP 271-83]. The pathogenesis of ischemic stroke is complex with multiple potential mechanisms. The carotid plaque is only one source of stroke, accounting to no more than 15-20% of cases [Petty GW, Brown, Jr, RD, Whisnant JP, Sicks JD, O'Fallon WM, Wiebers DO. Ischemic stroke subtypes. A Population-based study of incidence and risk factors. Stroke. 1999; 30:2513-16]. More frequently, infarcts are caused by more proximal sources of emboli- the heart and the aortic arch. The commonest causes of cardioembolic stroke are nonrheumatic (often called nonvalvular) atrial fibrillation, prosthetic valves, rheumatic heart disease (RHD), congestive heart failure and ischemic cardiomyopathy.

Recent population-based study from Rochester, Minn. found that the main identifiable subtype of ischemic stroke was cardioembolic with nearly 30% of cases, while all large-vessel cervical and intracranial atherosclerosis with stenosis altogether constituted about 16% [Petti et al., ibid. Further, often multiple mechanisms coexist] Caplan LR. Multiple potential risks for stroke. JAMA 2000; 283: 1479-801. Wilson and Jamieson reviewed their experience with patients who had high-grade internal carotid artery stenosis or occlusion and also had cardiac and aortic evaluation. Potential cardiac or aortic sources of emboli were present in 54% of patients; aortic arch plaques greater than 4 mm in diameter were found in 26% of patients with severe internal carotid artery occlusive disease [Wilson RG, Jamieson DG. Coexistence of cardiac and aortic sources of embolization and high-grade stenosis and occlusion of the internal carotid artery. J Stroke Cerebrovasc Dis. 2000; 9:27-30].

Prevention is clearly the most cost-effective approach to decreasing the burden of stroke. Available strategies to prevent stroke include Medical treatment, surgery (carotid endarterectomy) and carotid stenting.

Current Medical treatments include antiplatelet drugs (aspirin, ticlopidine, clopidogrel and dipyridamol) for presumed athreothrombotic origin. These treatments reduce the risk for recurrent ischemic event by no more than 15-20%. Anticoagulants like Warfarin for non valvular atrial fibrillation reduce the risk by 60% however even in carefully conducted and monitored clinical trial, substantial number of patients stopped anticoagulation [Hart RG, Benavente O, McBride R, Pearce La. Antithrombotic therapy to prevent stroke in patients with atrial fibrillation: a meta-analysis. Ann Intern Med 1999; 131:492-501].

Carotid endarterectomy was shown to be beneficial in selected cases of medium grade symptomatic and also in asymptomatic carotid stenosis >60%, whenever the complication rates are kept low [Chassin MR: Appropriate use of carotid endarterectomy (editorial). N Engl J Med 12998; 339:1468-71]. Nevertheless, a high proportion of recurrent stroke was not related to the large artery atherothrombotic disease, but to other causes including cardioembolism, as recently reported by the NASCET investigators [Barnett HJM, Gunton RW, Eliasziw M, et al. Causes and severity of ischemic stroke in patients with internal carotid artery stenosis. JAMA. 2000; 283:1429-36]. In fact, strokes related to cardioembolism tended to be more severe. The population of patients with carotid stenosis in ‘real life’ often includes patients with severe cardiac disease, concomitant protruding aortic arch atheroma, atrial fibrillation or congestive heart failure. The proportion of patients with such concomitant disease increases substantially in an elderly population. Thus the risk of recurrent cardioembolic stroke even in patients operated for carotid stenosis is estimated to be substantially higher [Barnett et al., ibid].

Carotid artery stenting has potential advantages of offering treatment to high-risk patients with carotid stenosis, lowering peri-procedural risk, decreasing costs and reducing patient inconvenience and discomfort. Preliminary results from clinical trials comparing carotid stenting to carotid endarterectomy have shown similar results [Major ongoing stroke trials. Stroke 2000 31: 557-2].

The approach to prevention of such a multi factorial complex syndrome as stroke has to be multifaceted Carotid angioplasty with stenting by itself does not address additional sources of emboli even after successful reduction of local stenosis. More efficient endovascular approaches to stroke prevention will have to take into account this complexity in cerebrovascular disease. In this context, an intravascular implant that addresses also prevention of emboli from proximal sources can be a valuable addition in the arsenal of the treating physician.

Introducing filtering means into blood vessels, in particular into veins, has been known for some time. However, filtering devices known in the art are designed for filtering blood flowing in the vena cava, and to stop embolic material having a diameter of the order of centimeters, and are unsuitable to deal with the arterial embolic material, with which this invention is concerned, the diameter of which is typically of the order of down to microns. Furthermore, the flow of blood in the veins does not resemble arterial flow in its hemodynamic properties. However, when considering the possible cerebral effects of even fine embolic material occluding an artery supplying blood to the brain, the consequences may be fatal or may cause irreversible brain damage.

In light of the short period of time during which brain tissue can survive without blood supply, there is significant importance to providing suitable means for preventing even small embolic material from entering the internal carotid artery, so as to avoid brain damage.

The size of the filaments that make up the filtering device, as well as the Porosity Index (as hereinafter defined), are of major importance in the device of the invention, as will be further explained below. In venous blood filters known in the art, in contrast, no particular attention has been paid to the size of the filaments. It should be noted that embolic material in venous blood is only made of blood clots, while in arterial blood it is necessary to deal with emboli including different materials, such as blood clots and atherosclerotic plaque debris, etc. On the one hand, in order to provide efficient filtering means, the filter should be of fine mesh. On the other hand, a fine mesh has a higher tendency toward occlusion.

It should also be noted that the flow ratio between the ICA and the ECA is about 3:1-4:1. This ratio also reflects the much higher risk of embolic material flowing into the ICA. The ECA, on the other hand, is a non-hazardous artery, because it supplies blood to superficial organs in the face and head, which are not life-supporting and which receive blood supply from collateral blood vessels. Therefore, embolic material reaching them does not cause substantial damage.

In two copending patent applications of the same applicant hereof, PCT/IL00/00145 and PCT/IL00/00147, there are described implantable stroke preventing devices. The devices of the present invention improve over the devices of the aforementioned copending patent applications in their ease and reduced cost of manufacturing, and in their flexibility of positioning.

It is known in the art to make braided stents and prostheses, for instance, from WO 97/16133, EP 804909, EP 895761 and WO 99/55256, which also describe methods of manufacturing the braided stents. Such braided stents present various advantages. However, they are all made for the purpose of preventing stenosis and for supporting blood vessels. The large mesh sizes employed, and the thickness and shape of the struts, do not make them suitable to be used as filtering means to deflect embolic material.

It is thus an object of the present invention to provide a diverting filter device suitable to prevent embolic material from reaching the brain.

It is another object of the invention to provide such a diverting device, that can be introduced in the vicinity of a bifurcation of an artery, particularly the bifurcation of the CCA into the ICA and the ECA, so as to divert the embolic material into the ECA.

It is yet another object of the invention to provide a method of manufacturing a diverting device according to the invention.

It is another object of the invention to provide a method for treating a patient known to suffer from embolic diseases, by selectively occluding the passage of embolic material into the internal carotid artery.

It is yet another object of the invention to provide a method for preventing conditions associated with embolic material.

Other objects of the invention will become apparent as the description proceeds.

### SUMMARY OF THE INVENTION

The present invention provides an implantable device (hereinafter referred to as “Diverting Filter”), which is an intravascular carotid artery stent-like device, designed specifically to prevent anterior circulation strokes from proximal embolic sources.

In a first aspect, the invention is directed to an implantable deflecting device for positioning in the vicinity of an arterial bifurcation for causing embolic material flowing toward a first branch of the bifurcation to be deflected into the second branch of the same bifurcation, comprising a deflecting filtering element suitable to deflect the flow of embolic material flowing toward said second branch, while filtering the blood flowing toward said first branch, said device comprising a braided tubular body having a contracted state with a first diameter, and an expanded state having a second diameter greater than said first diameter.

According to a preferred embodiment of the invention the implantable device is designed for positioning in the vicinity of a bifurcation of an artery leading to, or located in, the common carotid artery (CCA) on the one hand, and leading to a non-vital artery on the other hand, comprising a deflecting filtering element suitable to deflect the flow of embolic material flowing toward the CCA, into said non-vital artery, while filtering the blood flowing toward the CCA, said device comprising a braided tubular body having a contracted state with a first diameter, and an expanded state having a second diameter greater than said first diameter.

While balloon-expandable devices can be provided, it is usually preferred to use a device which is self-expandable. In a typical device according to the invention the length of a side of its opening after expansion is between 100-500 μm, preferably 200-400 μm, it has a diameter in the expanded state of 3-30 mm, it is made by braiding a number ranging from 40 to 160 of filaments together, and has a Porosity Index in the range 75%-95%, preferably 80%-90%.

Differently shaped and sized braided filaments can be employed. According to a preferred embodiment of the invention the braided filaments have a round cross-section having a diameter of 10-50 μm, preferably 20-40 μm. According to another preferred embodiment of the invention the braided filaments have a square cross-section before polishing of dimensions 10×10-50×50 μm, preferably 20×20-40×40 μm.

The deflecting device of the invention can be made of any suitable material. For instance, the filament can be made of a material selected from among 316L stainless steel, superelastic Nitinol, and mixtures of different metals and alloys.

In another aspect the invention encompasses a method for preventing the flow of embolic material flowing toward a first branch of an arterial bifurcation from entering into it, comprising implanting upstream to said bifurcation a deflecting filtering element suitable to deflect the flow of said embolic material into a second branch, said device comprising a braided tubular body having a contracted state with a first diameter, and an expanded state having a second diameter greater than said first diameter.

According to a preferred embodiment of the invention the method is a method for preventing the flow of embolic material flowing in the CCA from accessing the ICA, comprising implanting in the vicinity of a bifurcation of an artery leading to, or located in, the common carotid artery (CCA) on the one hand, and leading to a non-vital artery on the other hand, a deflecting filtering element suitable to deflect the flow of embolic material flowing toward the CCA, into said non-vital artery, while filtering the blood flowing toward the CCA, said device comprising a braided tubular body having a contracted state with a first diameter, and an expanded state having a second diameter greater than said first diameter.

According to a preferred embodiment of the invention the deflecting filtering element is implanted in the vicinity of the bifurcation of the common carotid artery (CCA) into the internal carotid artery (ICA) and the external carotid artery (ECA).

The invention further provides a method for preventing cerebralvascular diseases or their recurrence, comprising implanting in the vicinity of a bifurcation of an artery leading to, or located in, the common carotid artery (CCA) on the one hand, and leading to a non-vital artery on the other hand, a deflecting filtering element suitable to deflect the flow of embolic material flowing toward the CCA, into said non-vital artery, while filtering the blood flowing toward the CCA, said device comprising a braided tubular body having a contracted state with a first diameter, and an expanded state having a second diameter greater than said first diameter. In one particular embodiment, the deflecting filtering element is implanted in the vicinity of the bifurcation of the common carotid artery (CCA) into the internal carotid artery (ICA) and the external carotid arterv (ECA)

The device of the invention may have a diameter that varies along its longitudinal axis, so as to apply an essentially identical pressure at different locations within the artery. This is needed in many cases, because of the tapering nature of human arteries.

For instance, the diverting filter can be positioned in the carotid bifurcation, its proximal end in the common carotid artery (CCA) and the distal end in the external carotid artery thus providing filtration element at the ICA orifice and diverting embolic particles to the external carotid artery (ECA) territory. Another possible location is the brachiocephalic bifurcation, diverting particles to the right subclavian artery (the right hand) preventing the access to the right CCA.

The diverting filter may be combined with a conventional stent—e.g., for the treatment of bifurcation lesions, where a stent is positioned in the side branch and the diverting device in the main branch—wherein said conventional stent is deployed at the internal carotid artery and addresses local stenosis. The insertion and deployment techniques are similar to those employed in connection with a conventional stent. Bilateral procedures can be performed during the same session without increased risk, thus enabling deployment of bilateral carotid divertors. In addition, a diverting filter is similarly effective in diverting embolic material above a certain size, irrespective of the composition of the embolic material. Given that embolic matter may be composed of thrombotic material, platelet-fibrin particles, cholesterol, atheroma, or calcified particles such a mechanical diversion has an inherent advantage of being general to any embolic composition.

In a further aspect, the invention is directed to the prevention of the occurrence, or the recurrence, of cerebralvascular diseases, particularly of stroke, comprising preventing the flow of embolic material flowing in the CCA from accessing the ICA, by deflecting the flow of said embolic material into the ECA. Prevention of the cerebralvascular disease is achieved by implanting, permanently, in the vicinity of the bifurcation of the common carotid artery (CCA) into the internal carotid artery (ICA) and the external carotid artery (ECA), a deflecting device according to the invention.

It should be emphasized that while throughout this specification reference is made to the bifurcation of the CCA into the ICA, this is done for the sake of brevity only, but the invention is in no way limited to this specific location. The invention can be advantageously be exploited at any other suitable bifurcation of blood vessels as existing, for instance, in the leg.

All the above and other characteristics and advantages of the invention will be better understood through the following illustrative and non-limitative detailed description of preferred embodiments thereof.

### BRIEF DESCRIPTION OF THE DRAWINGS

In order to better understand the invention and to illustrate it in practice, non-limiting examples of some preferred embodiments will now be described, with reference to the accompanying drawings, in which:

FIG. 1A is a front view of a device in accordance with a preferred embodiment of the present invention;

FIG. 1B is a detailed view of an opening of the device of Fig. 1A, positioned within the artery;

FIG. 2 schematically illustrates the device of FIG. 1, located within the common and external carotid arteries.

FIG. 3 schematically illustrates the deployment of a self-expandable device;

FIG. 3A schematically shows the device of FIG. 1, in collapsed form (i.e., prior to expansion into the artery), on its way to reach the arterial bifurcation;

FIG. 3B schematically shows the device of FIG. 3A, during its expansion and positioning at the arterial bifurcation;

FIG. 3C shows a situation in which the device of FIG. 1 has been fully expanded, and the deploying equipment is free to be withdrawn;

FIG. 4 schematically illustrates a sheathed device, according to a preferred embodiment of the invention, in collapsed form, provided with a balloon for final expansion, as will be further explained hereinafter;

FIG. 5A shows in enlarged view a self-expandable device according to another preferred embodiment of the invention in its fully expanded position;

FIG. 5D is a detailed view of an opening of the device of FIG. 1A;

FIG. 5B shows the device of FIG. 6A constrained within a delivery device;

FIG. 5E is a detailed view of an opening of the device of FIG. 5B;

FIG. 5C shows the same device in place within the artery (the artery not being shown).

FIG. 5F is a detailed view of an opening of the device of FIG. 5C.

FIG. 6A illustrates the pattern of right and left common artery origin in which the arterial brachio-cephalic trunk and left common carotid artery are separated;

FIG. 6B illustrates the pattern of right and- left common artery origin in which the arterial brachio-cephalic trunk and left common carotid artery are joint;

FIG. 6C illustrates the pattern of right and left common artery origin in which exists four “independent” vessels;

FIG. 7 illustrates how the tapering of an artery leads to different diameters at the ends of the device;

FIG. 8A schematically illustrates a solution to the problem shown in FIG. 7;

FIG. 8B shows a non-cylindrical device according to another preferred embodiment of the invention;

FIG. 9 schematically illustrates a device with axially varying porosity index, according to a preferred embodiment of the invention;

FIG. 10A and B illustrates the making of devices with varying end, diameters, according to a preferred embodiment of the invention;

FIG. 11A is a front view of a device in accordance with a preferred embodiment of the present invention; and

FIG. 11B is the device of FIG. 11A with one end folded back upon itself.

### DETAILED DESCRIPTION OF PREFERRED EMBODIMENTS

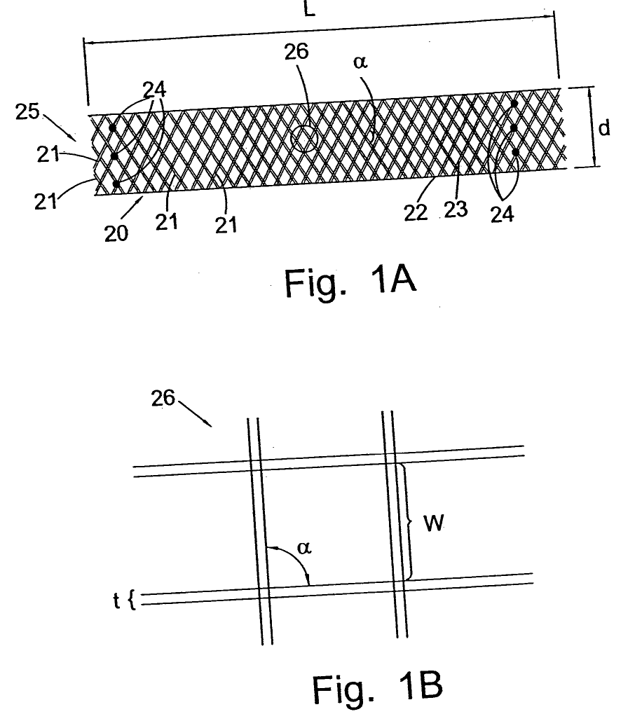

A device according to a preferred embodiment of the invention is schematically shown in FIG. 1A It consists of a substantially tubular body 20, that has been formed by braiding filaments 21, according to any technique known in the art of braiding tubular bodies, e.g., as described in any of the aforementioned patent documents. FIG. 1B is an enlargement of the area 26 of FIG. 1A. The braided deflecting device of the invention must possess critical dimensional characteristics, in order to function properly as a deflecting device. Said dimensional characteristics are substantially different from those of other tubular braided devices used for incorporation in the human body, such as stents. The following dimensional parameters should be observed:

Length of a side of the opening (or “window”) after expansion, i.e., between points 22 and 23 in FIG. 1A, “W” in FIG. 1B, between 100-500 μm, preferably 200-400 μm.

Diameter “d” of the device in the human body: 3-30 mm.

The dimensions of the braided diverting filter during manufacturing are, of course, a function of the final desired dimensions. The number of filaments to be braided is also a function of the dimensions of the window that it is desired to achieve, and are preferably in the range of 40-160 filaments

Porosity Index: 75%-95%, preferably 80%-90%.

Dimensions of the filament:

a) round cross-section (diameter “t”): 10-50 μm, preferably 20-40 μm;

b) square cross-section (before polishing): 10×10-50 ×50 μm, preferably 20×20-40×40 μm.

The filaments can be made of any suitable material, which is bio-compatible and which can be worked into a braid. According to a preferred embodiment of the invention, the filament is made of a material selected from among 316L stainless steel, tantalum, cobalt, and superelastic Nitinol, and any other suitable metal or metal combination. The filament can of course be coated with bio-compatible coatings [Ulrich Sigwart, “Endoluminal Stenting”, W. B. Saunders Companv Ltd., London, 1996]

The length “L” of the device will vary according to the intended location and use, and anatomical position, and is typically between 20 mm and 150 mm. According to a preferred embodiment of the invention the braided diverting filter is manufactured continuously, as an infinite sleeve which is then cut to the desired length. Cutting can be effected by any suitable method, e.g., by laser cutting at end 25 of the device. Rigid connection points 24 can be obtained, e.g., by welding or soldering.

After braiding is completed it is desirable, but not necessary, to anneal the device. Thermal annealing is preferred, which should be carried out at the temperature and for a period of time suitable for the chosen metal, e.g., at about 500° C. and for about 10 minutes for Nitinol. Other finishing processes, such as polishing, may be required, depending on the filaments employed and the manufacturing method.

As will be appreciated by the skilled person, the device made according to the invention has several advantages. The greatest advantage of the braided deflecting device according to the present invention, over other constructions,. is that it is possible to perform invasive procedures through it, by enlarging the openings and introducing a catheter. This procedure will cause a deformation of the filaments without leading to their rupture. This is an important feature, since the device is intended to occlude the opening into the ICA, and after it is positioned the ICA can only be reached through it.

However, other substantial advantages also exist. For instance, it is possible to employ perfectly rounded filaments, the resulting braided structure is flexible and, therefore, no harm will come to it from movement or pressure applied externally to the neck, and it is easy and inexpensive to mass-produce.

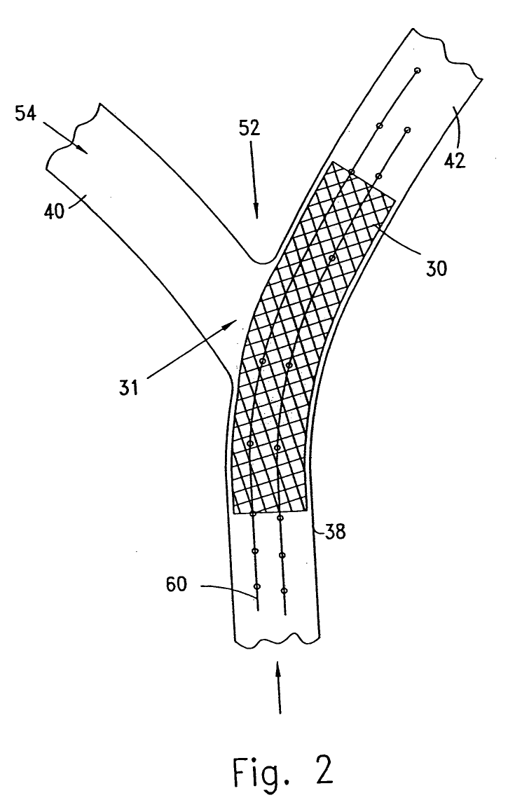

This is illustrated in FIG. 2, in which a device 30, made essentially as described with reference to FIG. 1, is seen in place in the ECA. FIG. 2 illustrates a carotid artery portion, in which the common carotid artery (CCA) is designated 38, the internal carotid artery (ICA) is designated 40, and the external carotid artery (ECA) is designated 42.

The deflecting device 30 is positioned within the bifurcation zone 52, opposite inlet 54 of ICA 40. The body of deflecting device 30 anchor against respective inner walls of the common carotid artery 38 and the external carotid artery 42, respectively. In this position, embolic material, which is schematically illustrated as particles flowing along flow lines 60 in FIG. 2, flows into the common carotid artery 38, and upon meeting portion 31 of the deflecting member they are prevented from entering the ICA 40, because their size is larger than the mesh of deflecting device 30, and they are thus deflected into the external carotid artery 42.

The deflecting device 30 has an essentially cylindrical shape with its body generally serving as an anchoring portion, anchoring portion is a portion of the device that firmly contacts the walls of the artery. Such contact causes a growth of the wall into the net of the devices, and strongly anchors it to the artery thus preventing its accidental displacement. The physiological processes leading to such anchoring are well known in the art, and will therefore not be discussed herein in detail, for the sake of brevity.

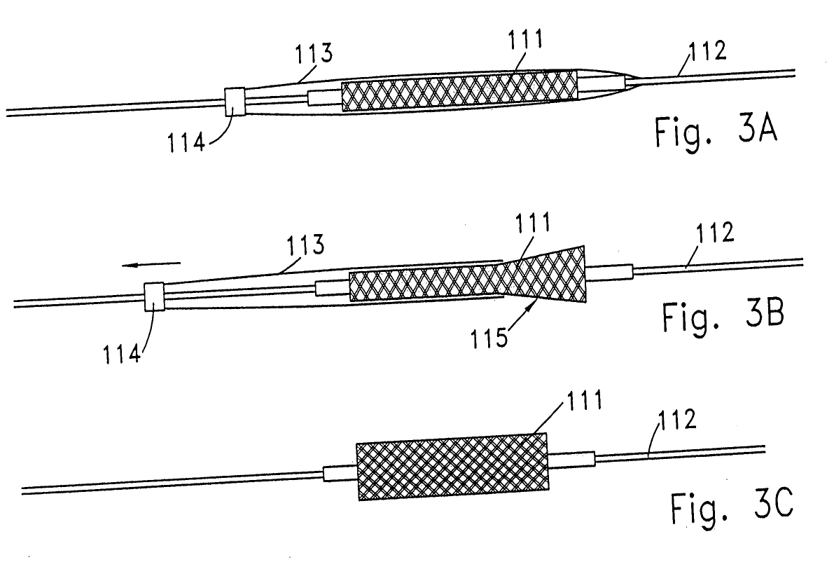

Introduction of the device of the invention and its deployment are schematically illustrated in FIG. 3. As will be apparent to the skilled person, using a self-expandable device is more appropriate in many cases, because of the great mobility of the neck of the patient.

FIG. 3A shows the diverting filter in folded state, FIG. 3B shows it during the first stage of expansion, and FIG. 3C shows it in fully expanded state. The diverting filter 111 is supported on a guide wire 112, which is used to introduce and guide it to the desired location. In its folded position, diverting filter 111 is covered with a covering envelope 113, which may be made of polymeric material, which keeps it in its folded state. Envelope 113 is connected to a retraction ring 114, which can be pulled away from diverting filter 111 by means not shown in the figure and well known to the skilled person. Looking now at FIG. 3B, when ring 114 is pulled away in the direction of the arrow, envelope 113 is pulled away with it, uncovering a portion of the diverting filter, indicated at 115. Since the envelope no longer obliges this portion 115 to remain in the folded position, and since the normal position of the diverting filter is expanded, this portion starts expanding to its natural, expanded state. This process is completed in FIG. 3C, when the envelope has been completely removed and the diverting filter is in its fully expanded position. Because elastic forces operate to keep the diverting filter expanded, its anchoring in its location is less susceptible of undesired displacement than balloon expanded stents. Of course, the guide wire is withdrawn from the patient after the positioning of the diverting filter and its expansion is completed, as in any other similar procedure.

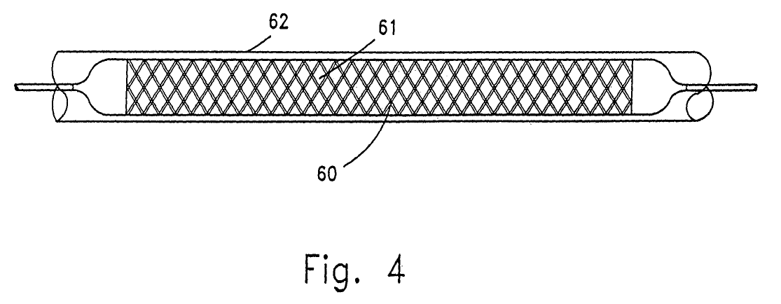

FIG. 4 shows a self-expandable device 60, according to a preferred embodiment of the invention, mounted on a balloon 61, and restrained by a restraining sleeve or sheath 62. It should be noted that balloon 61 is not used to expand the device, since the braided deflecting filter is self-expandable. Rather, it is used, once the device of the invention has been allowed to expand, in order to bring the device and the artery more closely into contact.

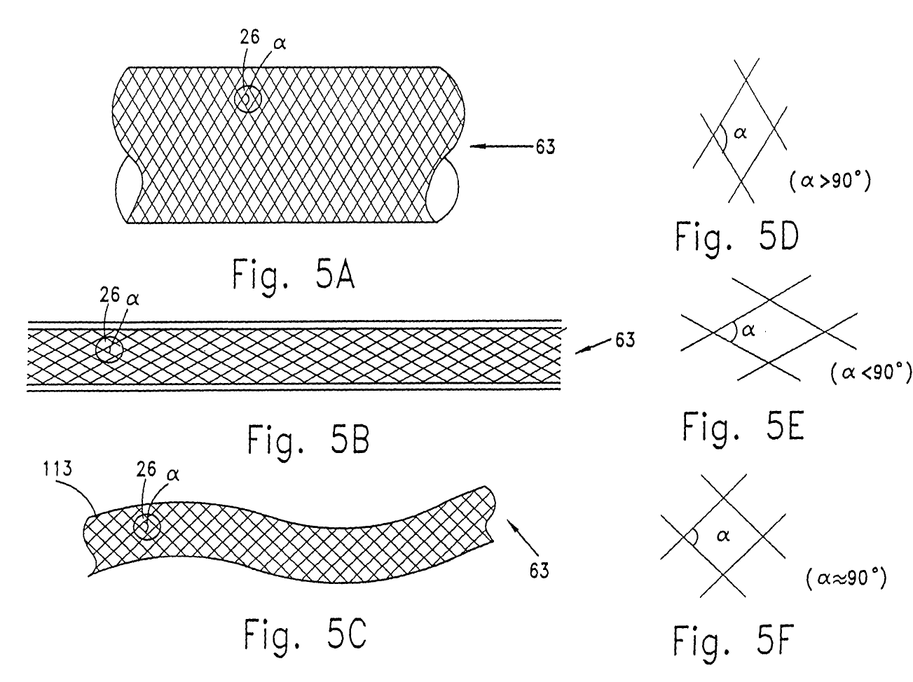

FIG. 5A shows a self-expandable device 63, according to another preferred embodiment of the invention. In FIG. 5A, the device is fully expanded and, as shown in FIG. 5D which is an enlargement of area 26 of FIG. 5A the angle α>90°. FIG. 5B is the same device constrained by a delivery device 113. In the constrained position, α>90°. as shown in FIG. 5E. FIG. 5C shows the same device in place in the artery (the artery not being shown). As shown in FIG. 5F,the angle α is in the vicinity of 90°.

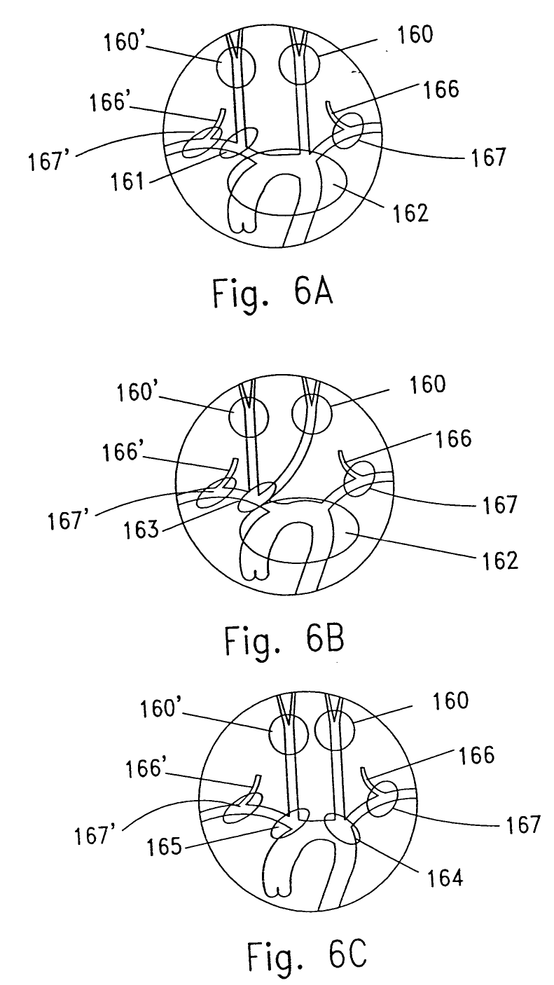

FIG. 6 illustrates different patterns of Right and Left common carotid artery origin. As will be appreciated by the skilled person, different persons may exhibit different patterns. Accordingly, the use of the deflecting filter of the invention is not limited to its positioning in the ICA-ECA bifurcation, which is described throughout this specification as the illustrative use, but the device of the invention can be positioned in any similar bifurcation, provided that it deflects the embolic material into an artery reaching non-vital organs, where the damage made by such material is minimal or non-existent.

FIG. 6A shows the most common pattern, in which the arterial brachio-cephalic trunk and the left common carotid are separated. In this situation, the diverting filter of the invention can be positioned at any one of the positions indicated by numerals 160, 160′, 161, 162, 167, or 167′.

FIG. 6B shows a pattern where the arterial brachio-cephalic trunk and the left common carotid artery are joint. In this situation, the diverting filter of the invention can be positioned at any one of the positions indicated by numerals 160, 160′, 162, 163, 167, or 167′.

FIG. 6C shows a pattern where four independent vessels exist. In this situation, the diverting filter of the invention can be positioned at any one of the positions indicated by numerals 160, 160′, 164, 165, 167, or 167′.

It should be noted that numeral 162 relates to the aortic arch. Positioning a deflecting device in the aortic arch will cover all blood vessels leading to the brain. This solution will also protect the vertebral arteries 166, 166′ from embolic material.

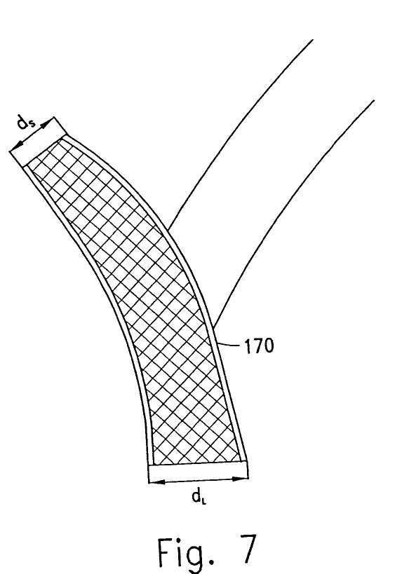

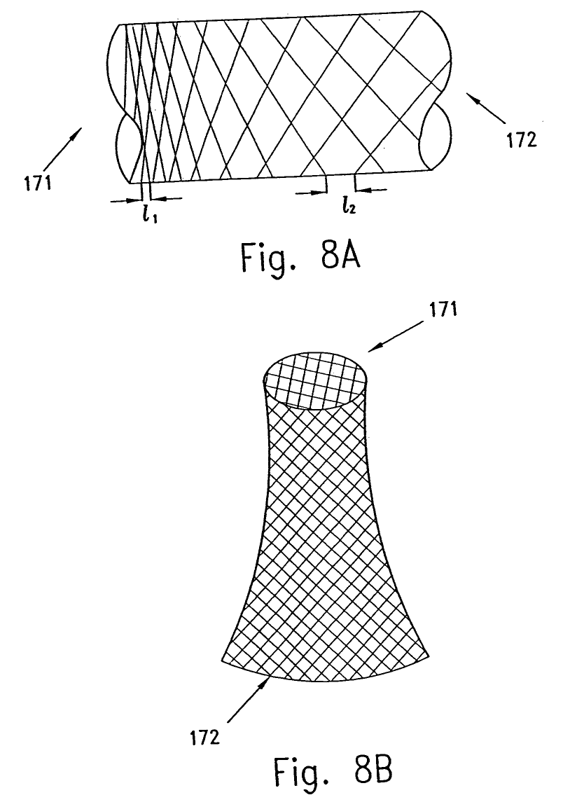

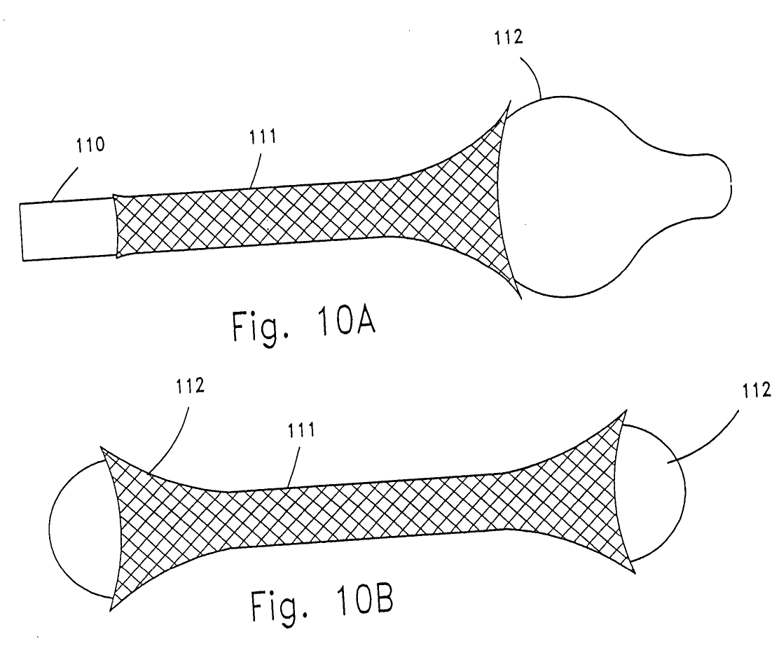

FIG. 7 illustrates a problem existing in many blood vessels, which is easily solved by the present invention. In the schematic illustration of FIG. 7 an artery 170, in which a deflecting filter of the invention is to be positioned, has different diameters at the two extremities of the device, where dₛ is smaller than dₗ. The difference can be of the order of 3-5 mm. As will be appreciated by the skilled person, if a constant diameter device is inserted into such a variable-diameter artery, this may result in a defective anchoring of the device at the larger diameter, and in a possible danger of dislocation under impact. In the braided device of the invention this problem can be easily overcome as schematically shown in FIG. 8A. This is done by varying the pitch of the filament turns at one end. Thus, in the example of FIG. 8A, the pitch l₁ is smaller than the pitch l₂. This will result in a stronger radial force at end 171 than at end 172 but will not alter the diameter if the device is allowed to open freely. However, if the device is placed in a variable-diameter artery, such as that of FIG. 7 in such a manner that end 172 of Fig. 8A corresponds to diameter dₛ of FIG. 7, the result will be a stronger radial force at end 171 which will hold the device firmly in position. FIG. 8B illustrates an alternative solution in which the expanded device is conical in shape as will be further discussed with reference to FIG. 10

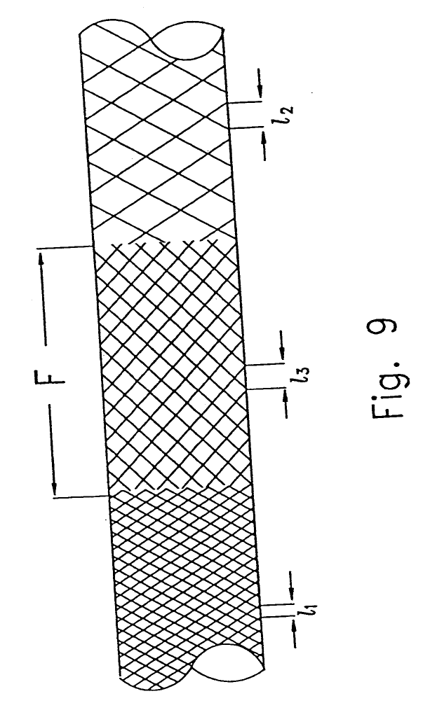

Varying the pitch of the filament turns also varies the porosity index. FIG. 9 schematically illustrates a device according to a preferred embodiment of the invention, which solves the problem illustrated in FIG. 7 and also maximizes the flow of filtered blood in the branch of the artery that the device is intended to protect. The device of FIG. 9 is constructed by taking the device of FIG. 8A and adding-a filtering zone “F” in which the pitch of the filaments is 3 which is greater than l₁ but smaller than l₂. As in the case of FIG. 8A, the pitch of the end sections of the device is chosen to increase the mechanical strength of the device and the anchoring with the wall of the arteries. The pitch in “F” is chosen such that when the device is in position in the artery, the angle between the filaments is in the vicinity of 90° thus maximizing the porosity index. FIG. 10 illustrates the making of devices with variable end diameters, according to a preferred embodiment of the invention. Looking at FIG. 10A, it is seen that the device of the invention, indicated by numeral 111, is braided on a mandrel 110, which has one enlarged end. 112. Braiding the filaments on said enlarged end 112 results in a larger diameter at the same end as illustrated in FIG. 8B. Similarly, in FIG. 10B the mandrel is enlarged at both ends, resulting in larger diameters at both ends of the device.

The device of the invention can be constructed in a way very similar to conventional stents. Typically, the braid is produced by combining one or more filamentary material, each of which passes over and under one or more other or same filamentary material in an interlace manner, as they are wound about a cylinder, cone or contoured mandrel, in a constant or variable orientation angles, porosity index and radii.

The braid may be removed from the mandrel after or during processing. It should be noted that braiding is a very well known process, and therefore it is not described herein in detail, for the sake of brevity.

It should be appreciated that the device of the invention presents certain characteristics that make it unique in the field of intraluminal devices. For instance, if compared to the Wallsten stent (which is a braided stent disclosed, e.g., in U.S. Pat. No. 4,655,771 and U.S. Pat. No. 5,061,275), which employs filaments of a typical diameter of 90 μm, while a typical device of the invention employs filaments having a diameter of between 10-50 μ, preferably about 20-40 μm, this leads to a decrease of two orders of magnitude in the mechanical strength of the resulting device. For this reason, it is usually desirable to employ initial α angles (see FIG. 5A′) of 140° or greater, preferably 140°-179°, more preferably 160°-170°, since the larger the value of this angle, the greater the radial force it provides, and hence the greater the strength of the resulting structure.

Additionally, the final angle α is between 80°-100°, preferably about 90° (FIG. 1B), because this is the angle that affords the maximal porosity index. The porosity index is defined by the relation:

$$1 - \frac{{S}_{m}}{{S}_{t}}$$

wherein Sₘ is the surface taken by the metal, and Sₜ is the total area, and wherein the ratio Sₘ/Sₜ is averaged over the surface, in each case where the ratio is not constant along the longitudinal axis of the device, as shown, for instance in Fig. 8A

Additionally, it should be noted that the Wallsten stent employs 24 filaments, while the device of the invention employs a much larger number of filaments, depending on the diameter of the device and upon the size of the windows (as shown in the example that follows). It is noteworthy that the Porosity Index of a typical Wallstent is about 80-85%, and although the device of the invention employs a much larger number of filaments, the Porosity Index of the device is about the same as that of the Wallsten stent.

Finally, it is most important to note the critical difference between the device of the invention and a regular stent. Because of the filtering nature of the deflecting device of the invention, in order for it to be effective it must possess openings of the order of magnitude of 100-500 μm, while a stent typically employs openings of the order of 1.2-1.5 mm.

Two characteristics of the invention, which must be improved in some applications, are its mechanical strength and radioopacity that result from the small diameter of the filaments, which are used to construct the device. The improvements can be made to these two properties in many different ways. According to a preferred embodiment of the invention, improved mechanical strength is imparted by using, together with the regular filaments of which the body of the device is made, a number, e.g., one or two stronger filaments, for instance, filaments having a diameter of 200 μm. Alternatively, strengthening rings can also be provided at the extremities of the device, or at a distance therefrom. Such thicker filaments and rings can also function as markers, to permit to locate the position of the device within the body.

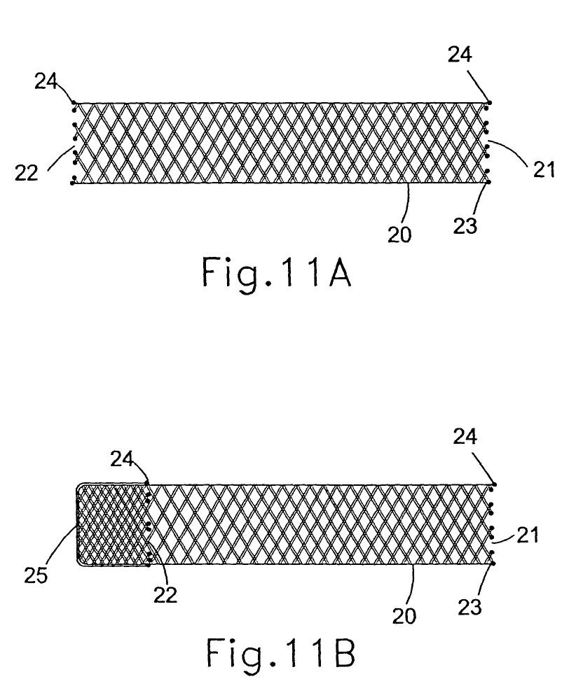

In another preferred embodiment of the invention the ends 21 and 22 of the device 20 of FIG. 11A are folded back upon themselves as one would roll up the cuffs of one's trousers. This operation can be performed at one end of the device, as illustrated in FIG. 11B where end 22 is rolled back forming new end 25, or at both ends if necessary. This method both increases the mechanical strength of the device and also provides a marker for locating the position of the device.

Other means of imparting additional mechanical strength to the device will be understood by the skilled person.

Further strengthening may be provided by welding or soldering overlapping filaments at selected points along the device, similarly to what is shown in the device (see 24, FIG. 1A). This welding limits the freedom of movement of the braid, and thus increases its mechanical strength.

Other means of solving the problem of radioopacity will be understood by the skilled person. For example, beads could be threaded onto the filaments at designated locations during the weaving process. Also if the braided material is cut with a laser, small beads are formed at the tip of each filament. If some or all of these are not removed, they will serve as markers for locating the device (as shown at 24 of FIG. 11). These methods and the others described above serve to aid a physician in the proper positioning of the device within the artery since the radio opaque markers are visible under radiographic equipment.

The filtering means (i.e., the “windows”) of the deflecting device should have dimensions of 100-500 μm, in order to effectively prevent the entrance of at least a major part of dangerous embolic material. The device of the invention must fulfill certain predetermined conditions that will be detailed hereinafter. The skilled person will of course be able to devise various devices, of different shapes and properties, which fulfill said conditions. When testing a device of the invention under physiological conditions, namely:

Reₐᵥ=200-500

BPM (beats per minute)=40-180

Womersley =2·7

wherein Reₐᵥ is the average Reynolds number, and Womersley is the dimensionless beat parameter;

the following conditions should preferably be met by the device of the invention:

1) Reₚᵣₒₓ between 0 and 4, preferably 1 or less (creeping or Stokes' flow)

2) 100 dyne/cm²>Shear Stress>2 dyne/cm²

wherein Reₚᵣₒₓ is the Reynolds number for the filaments of which the deflecting element is made, and the shear stress is measured at the device. As will be appreciated by the skilled person, the smaller the Reₚᵣₒₓ number the better. However, devices attaining larger Reₚᵣₒₓ numbers than indicated above may also be provided, and the invention is by no means limited to any specific Reₚᵣₒₓ number.

The invention is also concerned with a method of manufacturing the device of the invention that takes advantage of the shaped memory property of shape memory alloys, for example Nitinol. According to a preferred embodiment, the invention provides a method of manufacturing a device of the invention, comprising braiding the device with an angle between filaments different from the desired angle, changing the length of the device so as to obtain the desired angle, and applying a heat treatment suitable to provide the Nitinol filaments of the device with a shaped memory so that they will return to the desired angle when the compressed device is allowed to expand.

The invention will further be illustrated by the following example.

### EXAMPLE

Two deflecting devices, one made of Nitinol and the other of stainless steel, were made similarly to the device illustrated in FIG. 1, having the following characteristics:

Window size: 300 μ

Diameter of round filament: 35 μ

Porosity Index: 80%

Number of filaments: 96

Diameter: 7 mm.

The behavior of the device was compared with a Wallstent having the following characteristics:

Window size: 1250 μ

Diameter of round filament: 90 μ

Porosity Index: 85%

Number of filaments: 24

Diameter: 7 mm.

When measured by a method described by Wahn [Wahn A. N. Mechanical Strength. 2^{nd} Ed. New York: McGraw Hill; 1963 PP 241-254], The stainless steel device achieved 30% of the mechanical strength of the Wallstent while the Nitinol device achieved 13% of the mechanical strength of the Wallstent. Reducing the Porosity Index of the device of the invention, on the other hand, to 73% by increasing the diameter of the round filament to 50 μ increased the strength to 40% relative to the Wallstent in Nitinol, and to 90% in stainless steel. It is thus seen that the invention permits obviation of the strength problems inherent to the diameter of the filaments and other dimensions of the device of the invention, relative to a conventional stent.

The invention is useful in a variety of cases. Some illustrative indications are listed below:

1) Embolic strokes from proximal sources (e.g., mechanical heart valves, Afib, LVT, protruding AAA). These are:

Atrial fibrillation (2.5 million in the U.S.A. in 1999);

Mechanical heart valve (225,000 procedures performed annually in the U.S.);

Patients at high risk for recurrent embolism for a certain period (S.B.E.);

Patients at high risk for proximal emboli and absolute contraindications for anticoagulation;

Patients at high risk for proximal emboli failing best medical treatment.

2) In cases in which carotid stents are introduced to treat local stenosis, it is possible to introduce the device of this invention during the same procedure if there are concomitant high-risk proximal sources of emboli. These are, for instance:

Protruding Aortic arch atheroma (more than ⅓ of symptomatic patients);

Severe carotid stenosis with concomitant cardiac disease;

Severe carotid stenosis in patients undergoing heart surgery (5% on the statistical basis of 600,000 coronary bypass surgery)

While some preferred embodiments of the invention have been illustrated and described in the specification, it will be understood by a skilled artisan that it is not intended thereby to limit the disclosure of the invention in any way, but rather it is intended to cover all modifications and arrangements falling within the scope and the spirit of the present invention.

1. An implantable deflecting device for positioning in the vicinity of an arterial bifurcation for causing embolic material flowing toward a first branch of the bifurcation to be deflected into the second branch of the same bifurcation, comprising a deflecting filtering element suitable to deflect the flow of embolic material flowing toward said second branch, while filtering the blood flowing toward said first branch, said device comprising a braided tubular body having a contracted state with a first diameter, and an expanded state having a second diameter greater than said first diameter.

2. An implantable device according to claim 1, for positioning in the vicinity of an artery leading to, or located in, the common carotid artery (CCA) on the one hand, and leading to a non-vital artery on the other hand, comprising a deflecting filtering element suitable to deflect the flow of embolic material flowing toward the CCA, into said non-vital artery, while filtering the blood flowing toward the CCA, said device comprising a braided tubular body having a contracted state with a first diameter, and an expanded state having a second diameter greater than said first diameter.

3. A device according to claim 1 or 2, which is self-expandable.

4. An implantable deflecting device according to any one of claims 1 to 3, wherein the deflecting member generates a flow vector deflecting flow of embolic material into the ECA.

5. A device according to claim 1, wherein the length of a side of its opening after expansion is between 100-500 μm, preferably 200-400 μm.

6. A device according to claim 1, having a diameter in the expanded state of 3-30 mm.

7. A device according to claim 1, wherein the number of filaments braided is in the range of 40 to 160.

8. A device according to claim 1, having a Porosity Index of 80%-95%, preferably 75%-90%.

9. A device according to claim 1, wherein the braided filaments have a round cross-section having a diameter of 10-50 μm, preferably 20-40 μm.

10. A device according to claim 1, wherein the braided filaments have a square cross-section before polishing of dimensions 10×10-50×50 μm, preferably 20×20-40×40 μm.

11. A device according to claim 1, wherein the filament is made of a material selected from among 316L stainless steel, superelastic Nitinol, and mixtures of different metals and alloys.

12. A device according to claim 11, having a non-constant luminal diameter.

13. A device according to claim 12, wherein the luminal diameter at one extremity is greater than that at the other.

14. A device according to claim 13, wherein both extremities have a luminal diameter greater than its middle.

15. A device according to claim 1, having an axially non-constant porosity index.

16. A method for preventing the flow of embolic material flowing toward a first branch of an arterial bifurcation from entering into it, comprising implanting upstream to said bifurcation a deflecting filtering element suitable to deflect the flow of said embolic material into a second branch, said device comprising a braided tubular body having a contracted state with a first diameter, and an expanded state having a second diameter greater than said first diameter.

17. A method for preventing the flow of embolic material flowing in the CCA from accessing the ICA, comprising implanting in the vicinity of a bifurcation of an artery leading to, or located in, the common carotid artery (CCA) on the one hand, and leading to a non-vital artery on the other hand, a deflecting filtering element suitable to deflect the flow of embolic material flowing toward the CCA, into said non-vital artery, while filtering the blood flowing toward the CCA, said device comprising a braided tubular body having a contracted state with a first diameter, and an expanded state having a second diameter greater than said first diameter.

18. A method according to claim 17, wherein the deflecting filtering element is implanted, in the vicinity of the bifurcation of the common carotid artery (CCA) into the internal carotid artery (ICA) and the external carotid artery (ECA).

19. A method for preventing cerebralvascular diseases or their recurrence, comprising implanting in the vicinity of a bifurcation of an artery leading to, or located in, the common carotid artery (CCA) on the one hand, and leading to a non-vital artery on the other hand, a deflecting filtering element suitable to deflect the flow of embolic material flowing toward the CCA, into said non-vital artery, while filtering the blood flowing toward the CCA, said device comprising a braided tubular body having a contracted state with a first diameter, and an expanded state having a second diameter greater than said first diameter.

20. A method according to claim 19, wherein the deflecting filtering element is implanted in the vicinity of the bifurcation of the common carotid artery (CCA) into the internal carotid artery (ICA) and the external carotid artery (ECA)

21. A method according to claim 19 or 20, wherein the cerebralvascular disease is a stroke.

22. An implantable device according to claim 1, wherein the filament diameter is such that the Reynolds number for the wire is between 0 and 4, preferably 1 or less.

23. A device according to claim 1, wherein the angle between the filaments of the braiding varies along the longitudinal axis of the device.

24. A device according to claim 1, wherein the initial angle between the filaments of the braiding is 140° or greater.

25. A device according to claim 24, wherein the angle is between 140° and 179°.

26. A device according to claim 25, wherein the angle is between 160° and 170°.

27. A device according to claim 1, wherein the final angle, within the body, between the filaments of the braiding, is between 80° and 100°.

28. A device according to claim 27, wherein the final angle is about 90°.

29. A device according to claim 1, wherein the braiding is made of filaments of different diameter.

30. A device according to claim 1, which has essentially conical shape in expanded form.

31. A device according to claim 1 having varying diameters along its longitudinal axis, so as to apply an essentially identical pressure at different locations within the artery.

32. A device according to claim 1, comprising a plurality of locations near its ends at which crossing filaments have been soldered or welded together.

33. A device according to claim 1, further comprising one or more strengthening rings.

34. A device according to claim 1, in which one end of the device is folded back upon itself.

35. A device according to claim 1, in which both ends of the device are folded back upon themselves.

36. A device according to claim 1, in which beads' are threaded onto the filaments at designated locations during the weaving process.

37. A device according to claim 1, in which some or all of the beads that are formed at the tip of each fiber during the process of cutting the braid with a laser are not removed.

38. A method of manufacturing a device according to claim 1, comprising braiding the device with an angle between filaments larger than the desired angle, changing the length of the device so as to obtain the desired angle, and annealing the metal of the filaments so as to maintain the desired angle.

39. An implantable deflecting device for positioning in the vicinity of a bifurcation of an artery leading to, or located in, the common carotid artery (CCA) on the one hand, and leading to a non-vital artery on the other hand, essentially as described and illustrated.
